(19) United States
(12) Patent Application Publication
(10) Pub. No.: US 2025/0174311 A1
(43) Pub. Date: May 29, 2025

(54) METHODS FOR PREDICTING AND OPTIMIZING MIXTURES BETWEEN PETROLEUM PRODUCTS FOR PROCESSING IN THE SOLVENT ROUTE TO OBTAIN GROUP I LUBRICANT BASE OILS IN A PILOT PLAN

(71) Applicants: PETRÓLEO BRASILEIRO S.A. – PETROBRAS, Rio de Janeiro (BR); UNIVERSIDADE FEDERAL DO RIO DE JANEIRO – UFRJ, Rio de Janeiro (BR)

(72) Inventors: Anie Daniela MEDEIROS LIMA, Rio de Janeiro (BR); Felipo Doval ROJAS SOARES, Rio de Janeiro (BR); Maurício Bezerra DE SOUZA JÚNIOR, Rio de Janeiro (BR); Júlia Do Nascimento PEREIRA NOGUEIRA, Rio de Janeiro (BR); Argimiro Resende SECCHI, Rio de Janeiro (BR); Luis Carlos GOMES, Rio de Janeiro (BR)

(73) Assignees: PETRÓLEO BRASILEIRO S.A. – PETROBRAS, Rio de Janeiro, RJ (BR); UNIVERSIDADE FEDERAL DO RIO DE JANEIRO - UFRJ, Rio de Janeiro, RJ (BR)

(21) Appl. No.: 18/938,988

(22) Filed: Nov. 6, 2024

(30) Foreign Application Priority Data
Nov. 27, 2023 (BR) 1020230247350

(51) Int. Cl. G16C 20/30; G16C 20/70; G16C 20/90

(52) U.S. Cl. CPC G16C 20/30; G16C 20/70; G16C 20/90

(57) ABSTRACT

The present invention relates to data-driven methods for simulating the behavior of the solvent route. Specifically, a method is used in a simulator that estimates the behavior of the process. The methods comprise a prediction and an optimization method. The prediction method infers the properties of both the raffinate and the dewaxed from the properties of the feedstock and the manipulated variables. The optimization method defines which values of the manipulated variables generate the raffinate and the dewaxed with the desired properties.

### CROSS-REFERENCES TO RELATED APPLICATIONS

This application claims the benefit of and priority to Brazilian Patent Application No. 1020230247350 filed on Nov. 27, 2023, the contents of which are hereby incorporated by reference in their entirety for all purposes.

### FIELD OF THE INVENTION

The present invention applies to the activities of the oil and gas industry. Preferably, the present invention falls within the technical field of production units of group I lubricant base oils by means of a solvent route with the sequence of dearomatization and dewaxing processes, which use in their load portfolios, a paraffinic oil and a naphthenic oil.

### BACKGROUND OF THE INVENTION

It is currently known that the definition of the mixture occurs with extensive testing work in which the mixtures of distillate cuts of each distillation range, light neutral, medium neutral and heavy neutral, of petroleum (Light Arabic+naphthenic oil) are initially formulated in different proportions. Each formulated distillate is processed in the pilot dearomatization units and then dewaxing, in a search for adequate operating conditions for the specification of the dewaxed oil in terms of pour point and viscosity index. In the past, mass and solvent balances were used to determine the mixing limit between oils. This operation lasted about a year, when the mixture was recommended for an industrial test.

In this context, the production of group I lubricant base oils (sulfur content>0.38, 89<viscosity index<120 and saturates content<90%) by means of the solvent route involves only physical separation processes and hydrofinishing of the final products. The lubricant base oil has two striking characteristics, which are the viscosity index (VI) and the pour point. The VI represents the variation in viscosity with the working temperature, and higher values indicate smaller variations. The pour point indicates the lowest temperature at which the oil still flows, and the lower the better, since in countries with cold temperatures, it is necessary for the oil to flow when the engine starts. The solvent route begins with atmospheric distillation of the oil to separate the atmospheric residue (generally 370+° C.) from the fuels, followed by vacuum distillation, in which the distilled cuts and residue are removed, which will be refined and transformed into finished base oils. The subsequent process is usually a liquid-liquid extraction to remove low-viscosity index aromatic compounds. The refined product undergoes liquid-solid extraction to remove linear paraffins with high pour points. Finally, the final oils are hydrofinished to improve color and stability. To achieve the quality required for lubricant base oils, the oil must have a balanced composition of paraffinic compounds (higher VIs but higher pour points), naphthenic compounds (medium fluidity properties and VIs, but not removed in any process) and aromatic compounds (lower VIs but lower pour points). The absence of processes that produce chemical transformations also restricts the quality of the oil to be processed, since the final products have market requirements and ANP (Brazilian Petroleum Agency) regulatory standards (RANP 911/2022) in Brazil. The design of solvent route units usually involves Middle Eastern oils (Light Arabian, Kyrkuk, Basrahs, Kuwaiti and Iranian), which have a balanced composition, but tend to have a high sulfur content and are difficult to negotiate. Very naphthenic oils cannot be processed 100% in solvent route units because they do not allow the specification of finished oils.

Drawing a parallel with the technological resources currently used in the area, there are methods based on decision trees, such as Random Forests (RF) and Gradient Boosters (GB), which have shown superior performance both in the quality of the results obtained and in the computational time spent. RF is a machine learning algorithm presented by Breiman (BREIMAN, L. Random Forests. Machine Learning, v. 45, pages 5 to 32, 2001), composed of a group of estimators, also called decision trees (SPEISER, J. L., MILLER, M. E., TOOZE, J., et al. A comparison of random forest variable selection methods for classification prediction modeling. Expert Systems with Applications, volume 134, pages 93 to 101, 2019). GB was defined by Friedman (FRIEDMAN, J. H. Greedy function approximation: A gradient boosting machine. The Annals of Statistics, volume 29, number 5, pages 1189 to 1232, 2001) and, although it also commonly uses decision trees as estimators, it has a different algorithm from RF. The main difference between them is that RF is based on the concept of bagging, while GB uses the concept of boosting. The same author who introduced the RF algorithm proposed the definitions of decision trees and bagging (BREIMAN, L., FRIEDMAN, J., STONE, C., et al. Classfication and Regression Trees. Taylor & Francis, 1984. ISBN: 9780412048418; BREIMAN, L. Heuristics of instability and stabilization in model selection. The Annals of Statistics, volume 24, number 6, pages 2350 to 2383, 1996) a few years earlier. According to Zhou (ZHOU, Z. H. Ensemble methods: foundations and algorithms. CRC press, 2012), the concept of boosting was defined by Schapire (SCHAPIRE, R. E. The strength of weak learnability. Machine Learning volume 5, number 2, pages 197 to 227, 1990), while seeking to answer theoretical questions raised in the work of Kearns and Valiant (KEARNS, M. and VALIANT, L. G. Cryptographic limitations on learning Boolean formulae and finite automata. Proceedings of the Twenty-First Annual ACM Symposium on Theory of Computing (pages 433 to 444). New York, NY: ACM Press, 1989). Schapire proved that weaker learning methods could be boosted and become more robust methods. Basic definitions of decision trees, bagging, and boosting will be explained later for a better understanding of both techniques. RF and GB learning are performed in a supervised manner, that is, during training the expected outputs are shown to the models. In addition, they have a learning process known as shallow learning. Unlike deep learning, which typically corresponds to deep neural networks, most other ML techniques that are not brain-inspired are considered shallow learning (CHOLLET, F. Deep Learning with Python. Manning, 2017. ISBN: 9781617294433).

In regression problems, the predicted value will usually be within the range known during training. Models based on decision trees do not usually make extrapolations, so it is necessary to retrain the model for data outside the original known range. Similarly, small variations in the inputs do not generate variations in the outputs.

In view of the above, the present invention shows a methodology for estimating the percentage of mixture of these oils with the usual petroleum for the production of basic Light Arabic lubricants, aiming at the final specification of the basic oils, by means of modeling the database obtained from a pilot plant and simulation using a computer program.

In this sense, the present invention solves the aforementioned problem with methods for predicting and optimizing that predict the limit of 30% in a pilot plant for a mixture between petroleum A and petroleum B, using simulation.

Additionally, a retraining module was developed to improve the computer-implemented methods of the present invention, as new data are acquired, allowing the insertion of new data, retraining of models and the use of retrained models. Typically, dearomatization is the first process, although this is not always the case, the main objective of this process is the removal of aromatics and polar molecules. This process consists of liquid-liquid extraction using solvent. The usual solvents in this process are n-methyl-pyrrolidone (NMP), furfural and phenol. The removal of aromatics increases the VI, also promoting the reduction of the RI, reduction in density and improvement of oxidative stability. Conventional dewaxing is an expensive and energy-intensive process. The goal of dewaxing is to adjust the pour point by separating the n-paraffins from the solubility of non-paraffins in the cold solvent, fractional crystallization and filtration of the solution containing solid paraffins. This can be done in units using MEK, MEK/MIBK, MEK/Toluene solvents. Other alternatives are “propane” units that use liquefied propane as a solvent. Side effects include increased viscosity, increased density, increased sulfur and reduced VI.

After exploring several machine learning techniques that can be applied to modeling and predicting physicochemical properties, such as artificial neural networks (ANNs), duly discussed and applied both in predicting the properties of raffinate and dewaxed oils, and in optimizing the operating conditions of the dearomatization and dewaxing stages, methods based on decision trees were arrived at. Examples include Random Forests (RF) and Gradient Boosting (GB), which showed superior results. Pilot data from processing various proportions of Light Arabic with different crudes were used for modeling. In this way, it is possible to inform the program of a mixture of petroleum distillates that would be processed in the plant, and it will inform the operational conditions and results so that the operator can analyze the viability and decide on the best mixture.

### STATE OF THE ART

In the state of the art, there are approaches developed over time that seek to solve the technical problems described above. Specifically, observing the limitations of the state of the art, the present invention solves them by developing a method that simulates and a method that estimates the behavior of the process. The methods are prediction and optimization. The prediction method infers the properties of both the raffinate and the dewaxed from the properties of the load and the manipulated variables. While the optimization method defines which values of the manipulated variables generate the raffinate and the dewaxed with the desired properties.

The patent document US 20190002782 A1, for example, provides a lubricant base oil. In which the lubricant base oil has a low-temperature property determined using a stepwise regression of the peak values of carbon-13 nuclear magnetic resonance (NMR) spectroscopy. A method for selecting candidate lubricant base oils, or blends thereof, with acceptable low temperature performance is also provided. An online method for blending lubricant base oil and a finished lubricant is also provided.

U.S. Pat. No. 10,365,263 B1 describes methods and systems for predicting crude oil blend compatibility and optimizing blends to increase heavy crude oil processing. The method includes receiving ratios of crude oil physical parameters for crude oil blend optimization. The physical parameter ratios are based on Kinematic Viscosity (V), Sulfur(S), Carbon Residue (C), and American Petroleum Institute (API) gravity. Crude oil blend compatibility (K-model) is determined and generated using the physical parameter ratios. The K-model is developed by coefficients obtained by regression analysis between the physical parameter ratios of known crude oils and composite compatibility measures determined from multiple compatibility test results of known crude oils. The predicted crude oil blend compatibility can be used to optimize the processing of heavy crude oil.

The patent document US20130103627 A discloses a method for predicting the properties of crude oils by applying phase-jointed neural networks and characterized by determining the T2 NMR relaxation curve of an unknown crude oil and converting it into a logarithmic relaxation curve; selecting the values of the logarithmic relaxation curve located on a characterization grid; inputting the selected values as input data to a multilayer back-propagation neural network trained and optimized by means of genetic algorithms; predicting, by means of the trained and optimized neural network, the physicochemical factors of the unknown crude oil. The method comprises a process of training and optimizing a multilayer neural network of the backpropagation type. The method thus defined allows for the rapid prediction, without costly laboratory structures, of the most representative physical-chemical factors of crude oils, or alternatively the distillation curve of crude oils with an acceptable degree of approximation.

As can be seen, the present invention differs from the state of the art because it uses machine learning tools, uses models based on sets of trees, such as Random Forests and Gradient Boosting, and allows the models to be retrained, so that it is always possible to update the computer-implemented method, which will not become outdated. In short, the distinctive technical effects of the present invention, when applying the claimed method, include: (i) reduction of analyses performed in the laboratory, through the development of models that function as virtual sensors; (ii) support for the operation and control of the process, through the optimization of optimal operating conditions; (iii) support for decision-making in the formulation of lubricating oils, by proposing oil blends that meet the technical specifications for the quality of the final product; and (iv) maintenance and adaptation resources, with identification of new operating conditions, construction of updated databases, development of new models for the new conditions and evaluation of the new models, so that no methodology available in the state of the art provides the totality of effects provided by the present invention.

### BRIEF DESCRIPTION OF THE INVENTION

The present invention relates to a method for predicting blends between oils for processing in the solvent route, obtaining group I lubricant base oils, comprising: (a) creating a representative database of a production process for group I lubricant base oils, specifically of dearomatization and dewaxing steps; (b) statistically analyzing said database and variables of said process, determining a combination of input variables for a prediction of output variables, said variables being a yield of each said dearomatization and dewaxing step, and properties of a raffinate and a dewaxed product; (c) developing models from machine learning tools inferring said properties of raffinate and dewaxed product, such as density, refractive indexes and viscosity, as well as yield of each step, from properties of a load and operational variables; and (d) developing a retraining module, which is activated, as new data is acquired, allowing the insertion of new data, retraining of models, and the use of the retrained models as well as the original ones, which are no longer discarded if there is retraining. Wherein in step (d) activating said retraining module comprises: extracting statistics from old data, such as minimum, maximum, mean and standard deviation; analyzing new data, if the data are of the same type, assessing whether all variables exist; checking statistics made, whether values are within a minimum and a maximum of said old data; checking a mean and a covariance based on a Mahalabonis distance; and obtaining clean data without missing variables or different descriptive statistics, thus testing models with new data. Furthermore, step (d) comprises a retraining module composed of an alternative database, a valid database and an old database, in which said alternative is database composed of data whose analyzed descriptive statistics are different from the descriptive statistics of data used previously; in which said valid database is composed of data whose analyzed descriptive statistics are similar to the descriptive statistics of data used previously; and in which said old database is composed of initial data used in the development of the tool. Furthermore, in said alternative database of the retraining module of step (d), if the amount of data is equal to or greater than a number N, the amount of alternative data is evaluated. The method further comprises predicting a limit of 30% in a pilot plant for a mixture between a type of oil A and a type of oil B.

The present invention further relates to a method for optimizing mixtures between oils for processing in a solvent route to obtain group H lubricant base oils, comprising: (a) creating a representative database of a process for producing group I lubricant base oils, specifically of dearomatization and dewaxing steps; (b) statistically analyzing said database and variables of said process, determining a combination of input variables for an optimization of output variables of interest, that is, operating conditions of said process at each stage; (c) developing models from machine learning tools defining which values of the manipulated variables generate a raffinate and a dewaxed product with the desired properties, defined according to the characteristics of a product that is intended to be obtained, depending on its application; and (d) developing a retraining module, which is activated as new data is acquired, allowing the insertion of new data, retraining of models, and the use of the retrained models as well as the original ones, which are no longer discarded if there is retraining. Furthermore, if a mixture of oils results in valid operating conditions and yields, that is, physically and operationally viable values, it is considered that this mixture can be tested in the pilot plant with conditions similar to those optimized by the method.

Furthermore, the present invention relates to a computer-readable non-transitory storage medium comprising instructions stored therein, which when read by a computer, cause the computer to execute the steps of the method for predicting mixtures between petroleum products for processing in the solvent route to obtain group I lubricant base oils or of the method for optimizing mixtures between petroleum products for processing in the solvent route to obtain group I lubricant base oils.

### BRIEF DESCRIPTION OF THE FIGURES

The patent or application file contains at least one drawing executed in color. Copies of this patent or patent application publication with color drawing(s) will be provided by the Office upon request and payment of the necessary fee.

In order to complement this description and obtain a better understanding of the features of the present invention, and in accordance with a preferred embodiment thereof, a set of figures is shown in the appendix, where its preferred embodiment is represented in an exemplary, although not limitative, manner.

FIG. 1 shows a flowchart of the requested optimization method, with an initial strategy for the development of optimization models (green), input variables (orange) and output variables necessary to optimize the conditions (temperature and oil-solvent ratio of each stage). In optimization, the desired properties of the streams are used to obtain feedback on the appropriate operating conditions to achieve this result, in accordance with a preferred embodiment of the present invention.

FIG. 2 shows a flowchart of the requested prediction method, with an initial strategy for developing prediction models (green), input variables (orange) and output variables, so that, once the operating conditions are known, the model determines the expected yield of each stage, as well as the density and the refractive indexes and viscosity predicted for each stream, in accordance with a preferred embodiment of the present invention.

FIG. 3 shows a flowchart for building the databases, in accordance with step (d) of the requested methods, in accordance with a preferred embodiment of the present invention.

FIG. 4 shows a flowchart for retraining the models, in accordance with step (d) of the requested methods, in accordance with a preferred embodiment of the present invention.

FIG. 5 shows a simplified diagram of the liquid-liquid extraction bench test, as an experimental part for the methods claimed, for a given temperature, R=Raffinate, E=Extract, according to a preferred embodiment of the present invention.

FIG. 6 shows a block diagram of the dewaxing pilot units, as an experimental part for the methods claimed, according to a preferred embodiment of the present invention.

FIG. 7 shows a correlation matrix between the properties of the load and manipulated variables against the properties of the process output, as part of the statistical analysis for the methods claimed, according to a preferred embodiment of the present invention.

FIG. 8 shows a correlation matrix for the properties of the load and manipulated variables against themselves, as part of the statistical analysis for the methods claimed, according to a preferred embodiment of the present invention.

FIG. 9 shows the relationship between RSOdear and dR in the dearomatization spreadsheet data, as part of the statistical analysis for the requested methods, in accordance with a preferred embodiment of the present invention.

FIG. 10 shows the relationship between IRDP and RSOdew, specifically IRDP versus RSOdew in the dearomatization spreadsheet data, as part of the statistical analysis for the requested methods, according to a preferred embodiment of the present invention.

FIG. 11 shows a confusion matrix of the cut type classifier, as part of the statistical analysis for the requested methods, according to a preferred embodiment of the present invention.

FIG. 12 shows Tdear and dC stratified by cut, as part of the statistical analysis for the requested methods, according to a preferred embodiment of the present invention.

FIG. 13 shows prediction and residual graphs for the optimization method, as part of the statistical analysis for the requested methods, according to a preferred embodiment of the present invention.

FIG. 14 shows a current strategy for the requested optimization method (green), comprising the traditional input variables (orange), the output variables (other colors) and additional input variables, according to a preferred embodiment of the present invention.

FIG. 15 shows prediction and residual graphs for the prediction of dearomatization, of the requested prediction method, according to a preferred embodiment of the present invention.

FIG. 16 shows a current strategy for the requested prediction method of dearomatization and dewaxing (green), the traditional input variables (orange), the output variables (purple), intermediate variables (other colors) and additional input variables, where dR is an intermediate variable, being shown as an output variable and also used as an input variable for all dewaxing models, according to a preferred embodiment of the present invention.

FIG. 17 shows prediction and residual graphs for the claimed dewaxing prediction method, according to a preferred embodiment of the present invention.

FIG. 18 shows a current strategy for the claimed dearomatization and dewaxing prediction method (green), the traditional input variables (orange), the output variables (purple), intermediate variables (other colors) and additional input variables, where dR is an intermediate variable, shown as an output variable and also used as an input variable for all dewaxing models, according to a preferred embodiment of the present invention.

### DETAILED DESCRIPTION OF THE INVENTION

Specifically, the method of the present invention, in the case of optimization, focuses on determining what would be the ideal operating conditions to operate the pilot plant, aiming to obtain a certain desired value of IVDP (dewaxed viscosity index), from a cut with known properties. Thus, the variables to be calculated are: Tdear (dearomatization temperature), RSOdear (dearomatization oil solvent ratio), Tdew (dewaxing temperature), RSOdew (dewaxing oil solvent ratio). In the case of the prediction method, the objective is to calculate the properties of the raffinate and dewaxed, as well as the dearomatization and dewaxing yields: dR, IRR, IVR, YieldR, dDP, IRDP, IVDP, YieldDP, where R=raffinate and DP=dewaxed, d=density, IR=refractive index, Yield=yield and VI=viscosity index. Specifically, the method for optimizing mixtures between petroleum for processing in the solvent route to obtain group I lubricant base oils, of the present invention, comprises: (a) creating a database representative of the process; (b) analyzing the database and the process variables in order to determine which would be the best combination of input variables for optimizing the output variables of interest, in this case, the process operating conditions at each stage; (c) developing models using machine learning tools to define the values of the manipulated variables that generate the raffinate and dewaxed material with the desired properties; and (d) developing a retraining module that can be activated as new data is acquired, allowing the insertion of new data, the retraining of models, and the use of the retrained models as well as the original ones, which are not discarded if there is retraining.

The second preferred embodiment aims to predict the characteristics of the mixtures from the individual components, in order to be able to use the models without the need for a prior analysis of the load. IR and density can be inferred with a simple arithmetic mean, a result well established in the literature. Predicting viscosity and/or VI is more complicated, generally requiring calculation of the Gibbs free energy of the mixture or an interaction term (QIAN, J., ZHANG, J., ZHANG, Q., HUANG, Q., YANG, Z. Evaluation of Mathematic Models for Calculating Viscosity of Crude Oil Blends, Rio Pipeline Conference & Exposition 2005 Annals, 2004). Specifically, the method for predicting mixtures between petroleum products for processing in a solvent route to obtain group I lubricant base oils, of the present invention, comprises: (a) creating a database representative of the process; (b) analyzing the database and the process variables in order to determine which would be the best combination of input variables for predicting the output variables of interest, in this case, the yield of each stage and the properties of the raffinate and dewaxed; (c) developing models using machine learning tools to infer properties of the raffinate and dewaxed, based on load properties and operational variables; and (d) developing a retraining module that can be activated as new data is acquired, allowing the insertion of new data, the retraining of models, and the use of retrained models as well as the original ones, which are not discarded if there is retraining.

A preferred embodiment of the present invention has 2 models. Specifically, dearomatization with 2 manipulated variables: solvent-oil ratio (ROS) and temperature; and dewaxing with 3 manipulated variables: ROS, temperature and wash ratio. The method also comprises 3 oils: load, raffinate and dewaxed. All oils have density, IR and VI as characteristics. The load has the type of cut as an additional characteristic, while the raffinate and dewaxed have the respective yields of each process as characteristics.

Pilot data from processing various proportions of Light Arabic with different oils were used to model the method. The cuts are well separated by IVC, IRC and dC, so most models do not need to be informed of the cut explicitly, where c=cut.

The IR, density and VI measurements are standardized in ASTM, respectively D1218, D4052 and D2270 (ASTM-2016). The error of the measurements of each variable for measurements made by different people is given in the ASTM standards. The yield does not have an associated error because it is not a standardized measurement, but the yield error is estimated to be ±1.

In general, for modeling purposes, it is assumed that manipulated variables have negligible errors, but for simulation purposes, an acceptable range is sought in which the model error does not significantly interfere with the result.

The correlation analysis is based on calculating the correlation between each variable that was made for each cut. The analysis was divided by cuts to avoid the Yule-Simpson effect in which trends that appear in parts of the data are lost when the whole is analyzed (SIMPSON, E. H. The Interpretation of Interaction in Contingency Tables. Journal of the Royal Statistical Society, Series B, number 13, pages 238 to 241, 1951).

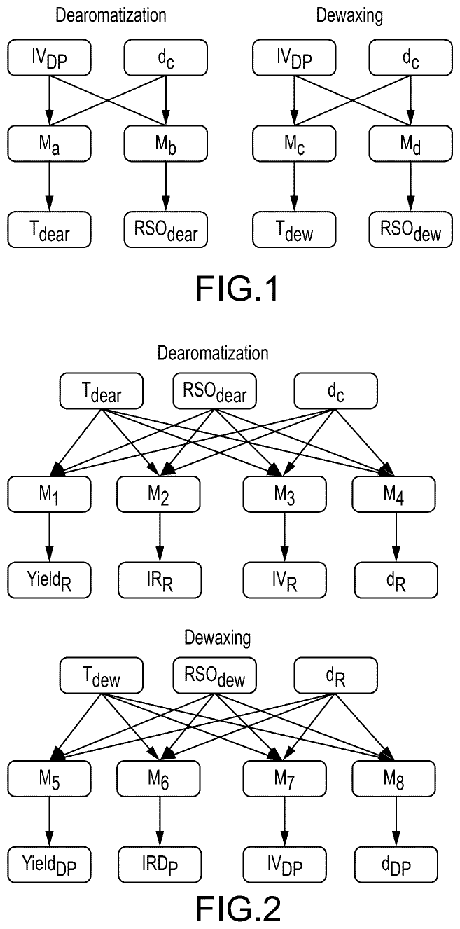

Specifically, the chemical process variables are inputs and outputs of the methods of the present invention, as illustrated in FIGS. 1 and 2. Phenomenologically, the properties of the raffinate and dewaxed streams, such as density and refractive indices and viscosity, as well as the yield of each step, depend on the temperature at which each step of the process is performed, as well as the solvent-to-oil ratio used in it.

The methods of the present invention have the capacity to incorporate more data, if they are made available and statistically relevant, as treated by the retraining module. In other words, the methodology is adaptable if the database can be expanded in the future, increasing the current validity interval. Furthermore, if different types of samples are used in the future (for example: analyses from different sources of oil not covered by the database available when the methods were developed), these will represent cases for which the developed and currently available methods were not trained to perform prediction and optimization. Therefore, the retraining module represents flexibility in the methodology, allowing these new samples to be added to the database, and new models that encompass all old and new knowledge can be developed. Consequently, prediction and optimization for new cases become possible.

In another embodiment of the present invention, a non-transitory computer-readable medium is provided. The medium may be, for example, a memory, a flash memory, a hard disk, a compact disk, or any other device capable of storing computer instructions. When the readable medium of the present embodiment is read by a computer, the computer is enabled to perform the method for predicting mixtures between petroleum products for processing in the solvent route to obtain group I lubricant base oils or the method for optimizing mixtures between petroleum products for processing in the solvent route to obtain group I lubricant base oils.

### Example of Embodiment/Tests/Results

Tests were performed for analyses both to evaluate how the properties of the load are influenced by the cut, and to predict the VI of the mixture from the data of the individual components.

The first analysis was performed to better elucidate the behavior of the models and to explain why some do not need to receive the cut explicitly as an input variable. Basically, the cut is implicit in the properties of the load, density and VI. A small RF classification model was tested, which attempts to infer the sample cut type from the input data, thus showing that the model can infer the cut type from the load property data. Additionally, the stratification of certain operating conditions in terms of the cut type was analyzed.

The second analysis predicts the characteristics of the mixtures from the individual components, in order to be able to use the models without requiring a prior analysis of the load. IR and density can be inferred with simple arithmetic mean, a result well established in the literature, as previously mentioned. Predicting viscosity and/or VI is more complicated, requiring the calculation of the Gibbs free energy of the mixture or an interaction term (Qian et al., 2004).

In the case of the optimization method of the present invention, whose focus, as mentioned, is to determine what would be the ideal operating conditions to operate the pilot plant, to obtain a certain desired value of IVDP, from a cut with known properties, the variables to be calculated are: Tdear, RSOdear, Tdew, RSOdew.

Initially, the models were developed based on one of the main approaches used previously: the use of dC and IVDP to calculate each of the operating conditions of interest, as observed in the structure of FIG. 1.

In order to improve the test results, models with different input variables were developed to calculate each of the variables of interest. IVDP was added or replaced by expanded terms, such as 1/IVDP, IVDP2 and log (IVDP). In addition, the addition of the mixture percentage (%), IVC, and both simultaneously were tested. For Tdew and RSOdear, variables that are more difficult to predict, the type of cut (Cut) was also tested as input.

Each of these models was created using 120 samples. This is the amount that has all the necessary input and output variables available. For modeling, the samples were divided between 80% for training and 20% for testing. The validation fraction was optimized between 10% and 50% of the training data.

Since the four output variables of interest usually have integer values, or very concentrated, it was necessary to insert low noise only in the training data. The objective was to help the machine learning techniques to make prediction, without generating concentrated or biased results. Normal noises of optimized mean and deviation were inserted, with a search range described in Table 1.

TABLE 1

| | | |
|---|---|---|
| Mean | Tdear e RSOdear | −0.5 a 0.5 |
| | Tdew e RSOdew | −0.2 a 0.2 |
| Standard deviation | Tdear e RSOdear | 0 a 0.5 |
| | Tdew e RSOdew | 0 a 0.2 |

The techniques tested were RF and GB. The hyperparameters were optimized by maximizing the R² of the validation data after training. For each model, optimized and trained independently, 500 iterations were performed in search of the most appropriate hyperparameters. Table 2 below shows this search range.

TABLE 2

| | |
|---|---|
| Number of trees | 1 a 100 |
| Maximum Depth | 5 a 10 |
| Minimum number of Samples | 1 a 10 |
| Generate Node | |

New training was performed with the already optimized hyperparameters. Subsequently, the test data, previously unpublished for the models, were evaluated in order to identify which was the best model for each variable of interest.

In the case of the prediction method, as previously mentioned, the objective is to calculate the properties of the raffinate and dewaxed products, as well as the dearomatization and dewaxing yields: dR, IRR, IVR, YieldR, dDP, IRDP, IVDP, YieldDP.

The methods were developed based on the main approach: using the stage load density (dC or dR), together with its operating conditions (Tdear and RSOdear or Tdew and RSOdew) to calculate each of the desired properties, as observed in the structure of FIG. 2.

Based on some observations made in the data behavior, and seeking models with increasingly better results, some expanded terms were added or replaced as input variables. Some examples are 1/RSO, RSO², log(RSO), T*RSO and T*log(RSO) of the stage being modeled. In the case of YieldDP and IVDP, variables that are more difficult to predict, the mixing percentage (%) was also tested as input.

Unlike the optimization method, the number of samples available for each model was different. They are shown in Table 3 below.

TABLE 3

| | |
|---|---|
| YieldR | 264 |
| d_{R} | 265 |
| IR_{R} | 265 |
| IV_{R} | 210 |
| YieldDP | 115 |
| d_{DP} | 121 |
| IR_{DP} | 121 |
| IV_{DP} | 121 |

In a similar way to that described in the optimization method, 80% of the samples were separated for training, and the fraction of them to be used in validation was optimized between 10% and 50%. The same noise insertion technique was applied, but this time only in the IVDP prediction. Only this output variable is composed of integers and aggregated around certain values. The mean was optimized in the interval [−0.2, 0.2] and the standard deviation was sought between [0, 0.2].

The same techniques were applied, and the hyperparameters were also optimized, maximizing the R² of the validation. The same search intervals shown in Table 2 were used, as well as the 500 interactions. Once again, the model trained with the appropriate hyperparameters evaluated the test data to determine the best model developed for each variable.

In another preferred embodiment of the present invention, a graphical interface was created using the PySimpleGUI 4.44.0 library and the transformation of the script into an executable was performed using the PyInstaller 4.3 library. The joblib 0.14.0 library was used to load models and the NumPy 1.17.3 library was used for data manipulation and calculations.

All codes were developed in Python 3, using Visual Studio Code 1.39.2 as the integrated development environment (IDE). The RF, GB and MVS models were created using the RandomForestRegressor, GradientBoostingRegressor and SVR functions available in the scikit-learn 0.23.2 library, their hyperparameters were optimized using the Optuna 2.4.0 library and the joblib 0.14.0 library was used to save and charge the models. Auxiliary libraries such as NumPy 1.17.3, pandas 0.25.3 and matplotlib 3.1.0 (Hunter, 2007) were used for data manipulation, calculations and generation of graphical results.

The methodology was designed to be implemented on a computer, created to provide two distinct methods: the prediction method and the optimization method. The first receives from the user the data necessary to calculate the properties of the raffinate and dewaxed, in addition to the yield of each stage of the process, in which the prediction models are activated and used.

In the case of the optimization method, the user will provide the data needed to calculate the ideal operating conditions for dearomatization and dewaxing. The optimization method models are activated and used. Another feature of this method is to use these calculated conditions as input variables for the prediction models developed, calculating the yield of the stages and the properties of the products in a secondary manner.

In both methods, dR can be used as an internal variable, since it is predicted, and also applied as an input variable in some models. The user is given the option of using the dR value as predicted by the available model (default option), or of providing this value to the program, if it is available.

In this way, it is possible to inform the program of a mixture of petroleum distillates that would be processed in the plant, and it will inform the operating conditions and results so that the operator can analyze the feasibility and decide on the best mixture.

One of the objectives of the interface is to maintain a calculation memory, in order to facilitate the use and storage of the results of the analyses performed in the software. The output log.txt file stores a summary of each analysis, which is updated each time the user activates the buttons to calculate or optimize. An output text is generated and added to the end of the output log.txt file, containing the date and time of the recording, along with information on whether a simulation or an optimization method was performed.

In addition, a summary of the data used as input and the results calculated to generate the calculation memory is printed. If there is a problem, such as a variable out of range, this warning will be included in this text to remind the user that the results of this analysis may not be reliable.

A retraining module was developed to allow continuous improvement of the methods. It is capable of registering new types of distillate cut previously unknown to the software, receiving new data, generating and evaluating new models and, depending on the results, applying them to new predictions or optimizations.

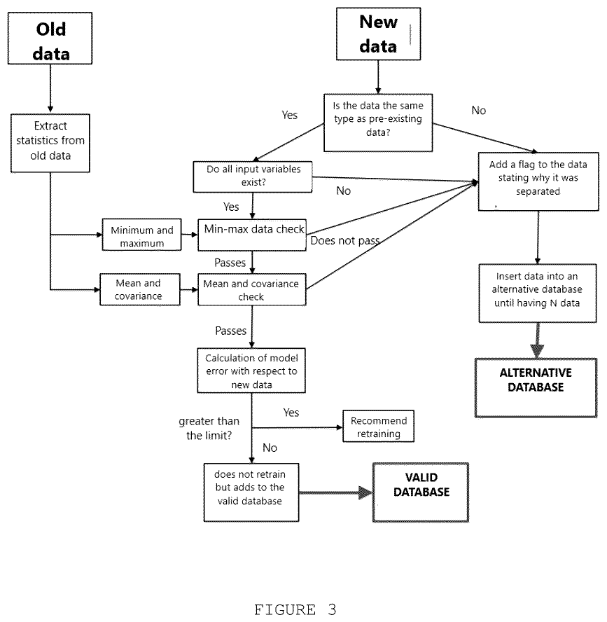

The retraining methodology focuses on how to systematically create and maintain a database that is useful for retraining. The methodology was developed, assuming a possible process deviation, that the oils to be tested may come from different cuts or have characteristics that are very different from those seen previously, as seen in FIG. 3.

The first step is to extract statistics from the old data: minimum, maximum, mean and standard deviation. Then the analysis of the new data begins. First step: are the data of the same type as the existing data? For example: Is there a new cut? If the data is of the same type, it is assessed whether all the variables exist. Sometimes, some data such as IVC or yields are not recorded.

Next, statistical checks are performed. First, it is checked whether the data is within the minimum and maximum of the old data. This is important for decision tree-based models, as they treat data beyond the minimum and maximum as if it were the minimum and maximum. For example: a model trained with data between 0 and 1 will treat a data with a value of 10 as if it had a value of 1. Then, a mean and covariance check is performed based on the Mahalabonis distance. If the Mahalabonis distance is too large, it means that the distribution of the data diverges greatly from the original data. The Mahalabonis distance is defined as d=\sqrt{(x−μ)*Σ⁻¹*(x−μ)}, where d is the Mahalabonis distance, Σ is the covariance matrix, and μ is the mean of the variables. This check is included mainly due to the strong correlation between the IRs and the densities, since the loss of this correlation indicates a divergence in the behavior of the oil.

With the clean data, the models are tested with the new data. If the model agrees with the new data within a certain limit, there is no reason to retrain. Absolute agreement of the data with the model is unlikely and retraining can increase the uncertainty of the model. The data is then saved in a new database for future use. This database is called the “valid database”. If the model error is greater than the limit, retraining is recommended, and the data is saved again in the valid database.

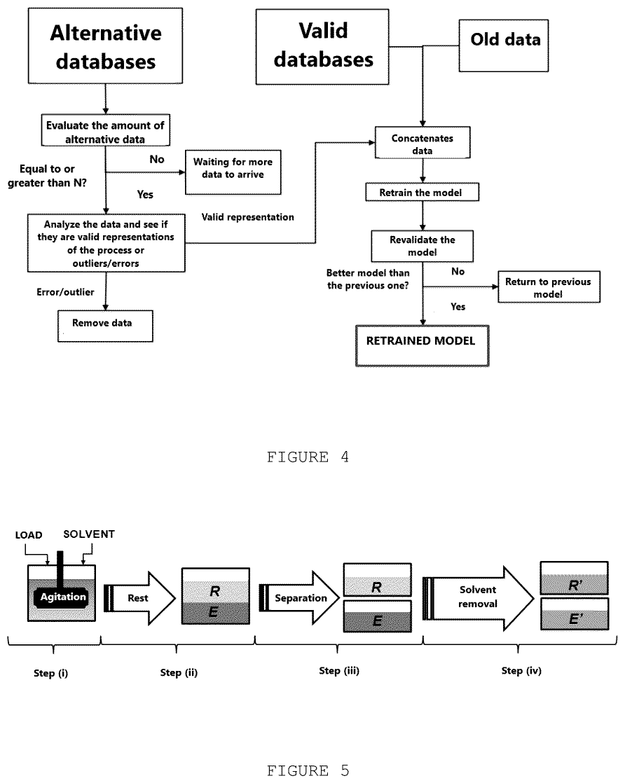

If the new data does not pass any stage of the check, a warning is added informing why the data did not pass. This data is stored in a database called “alternative database” until it has a significant amount N. After the databases are organized, the methodology moves on to the retraining stage. The functioning of the proposed retraining part can be seen in FIG. 4.

If the methodology recommends retraining, the first step is to reevaluate the alternative database. If it does not have enough data, new data is expected. If the data is sufficient, it is analyzed whether the data are really outliers, coming from runs with problems, or whether they are valid representations of the process. A common problem that can occur is that Excel confuses “.” and “,” depending on whether it is configured in Portuguese or English. This causes data to be remembered not as a decimal but as thousands, in which 1.5 becomes 1500, thus not passing the min-max filter. Data that definitely cannot be used is discarded and data that provides a valid representation is concatenated with the old data and the valid database.

With the data ready, the models are retrained following the same hyperparameters as the original model and revalidated. If the model is better than the previous one, the new model is saved and will be used for future predictions. If it is worse, the method returns to the previous model. The new models developed can be used in specific screens within the retraining module to perform predictions and optimizations.

For revalidation, it is recommended to use cross-validation, in which the model is continuously trained and tested with different data until all data has been tested; or holdout, in which only one division between training and testing is made. A residue analysis is also recommended.

If the oil blend proposed for the simulation and optimization methods results in valid operating conditions and yields, it is considered that this blend can be tested in a pilot plant with conditions similar to those optimized by the methodology.

In general, the pilot solvent route of the tests performed has the basic steps: (i) dearomatization, (ii) dewaxing. The load of this route is usually a cut obtained from vacuum distillation or a deasphalted oil. Possible loads of this process are light neutral, medium neutral, heavy neutral, spindle distillate and deasphalted oil cuts. The pilot process consists of bench dearomatization. The extracted raffinate undergoes dewaxing in order the final cuts meet minimum pour point and viscosity index characteristics: light neutral, VI≥100 and pour point≤−6° C.; medium neutral and heavy neutral, VI≥95 and pour point≤−3° C.; spindle, VI≥100 and pour point<−6° C.

It is known that bench dearomatization is performed by means of liquid-liquid extraction. The principle of this process is to place a liquid mixture of two or more components in contact with a solvent, which has a greater affinity for one of the components of the mixture, thus creating two immiscible liquid phases. The difference in affinity or solubility is a function of the chemical nature of the molecules.

Basically, the liquid-liquid extraction bench test consists of the following steps: (I) mixing and stirring the load with the solvent at a temperature of at least 20° C., below the critical solubility temperature; (II) letting the mixture rest for a given time until two immiscible liquid phases are formed; (III) separating the two immiscible liquid phases into different containers; and (IV) removing the solvent from each liquid phase, as can be seen in FIG. 5, in a simplified form, the liquid-liquid extraction bench test for a given temperature.

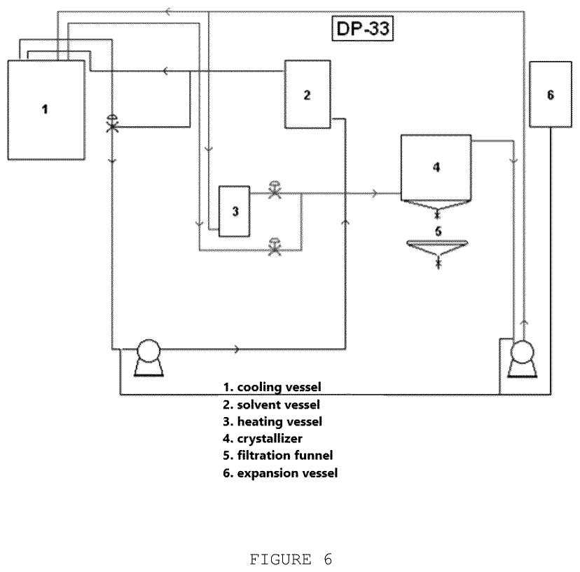

The unit feed is composed of load (distillates or deasphalted) and solvent, and is separated into two phases: (i) raffinate phase—poor in solvent, containing mainly oil components, (ii) extract phase—rich in solvent, containing mainly aromatic and polar components. The main operational variables that govern bench dearomatization are (NOGUEIRA W S, MORAES M F, 1993. Comparative studies with phenol, furfural and n-methyl-2-pyrrolidone as solvent for lube oils extraction. In Proceedings: International Solvent Extraction Conference, London, England; LYNCH, T. R. Process Chemistry of Lubricant Base Stocks. CRC Press, Boca Raton, Florida, 2008): (I) solvent (the criteria of density, viscosity, volatility, chemical stability, toxicity, corrosivity, availability and cost must be observed); (II) solvent-oil ratio (ROS) (an increase in the solvent-oil ratio (ROS) or solvent-load ratio leads to a decrease in yield, since more material is extracted, but an improvement in the quality of the raffinate), although this effect is not linear and is more sensitive to low ROSs; (III) extraction temperature (T), in which an increase in temperature increases the solubility of the load in the solvent, promotes greater extraction, but reduces selectivity. Some compounds solubilized in the extract could compose the raffinate. Therefore, if the extract phase is the phase of interest, an increase in temperature generally increases yield but decreases quality due to loss of selectivity. If the raffinate phase is the phase of interest, an increase in temperature generally improves quality but decreases yield. The dewaxing pilot unit is composed of six main pieces of equipment, as shown in FIG. 6.

The process is semi-automatic, since the procedure consists of: a) weighing in a volumetric flask, inside a hood, the quantity of sample (refined oil) and solvent that will be used in the run; b) heating this sample in an electric blanket, also inside a hood, until the temperature at which the load begins to crystallize; c) transferring this heated flask to the crystallizer of the pilot unit, which must be at the same temperature; d) starting the supervisory system of the unit to perform process control.

Since the pilot units operate in a wide temperature range, including negative temperatures, ethanol was chosen as the thermal fluid for the test. This is fed by the expansion vessel and distributed throughout the unit by centrifugal pumps, ensuring the required temperature at each point of the process. The compressed air flows are regulated for the correct operation of the pneumatic control valves that will act on the process temperatures. The cooling vessel is kept at −70° C. with dry ice and silicone, so that during the process it reaches temperatures of around −30° C. The process begins by defining the following parameters in the control supervisory unit: the temperature to start crystallization (T=+70 to +60° C.), the temperature to cool the washing solvent vessel (T=+20 to −30° C.) and the cooling ramp that must be followed during crystallization. Once these variables have been defined, the heating vessel begins to heat the fluid in the line in order to guarantee the desired temperature in the crystallization vessel. During this heating process, the washing solvent is transferred to the solvent vessel, while, in a parallel circuit to the heating circuit and always with the control valves operating, the solvent vessel begins to cool down to the filtration temperature of the load, also defined in the supervisory unit by the cooling ramp.

Upon reaching the filtration temperature of the batch, a vacuum is applied to the filtration line and, through a Buchner funnel fitted with a filter cloth, filtration begins to separate the oil from the crystallized paraffin. During this stage, the cake is washed by adding the washing solvent (previously cooled in the solvent vessel to the filtration temperature (T=−30° C. to +25° C.)).

After washing the cake, the filtrate is collected in kitassatos. During this collection, the filtration rate associated with the filter cloth used can be determined by attaching a collection balloon, determining the rate before starting to wash the cake. This parameter is measured manually with the aid of a stopwatch, and its main variables are the solvent-oil ratio and the filtration temperature. The distillates and deasphalted oils were processed in this route and the data were used to model the methods of the present invention.

Thus, the methods of the present invention have dearomatization with 2 manipulated variables: solvent-oil ratio (ROS) and temperature; and dewaxing with 3 manipulated variables: ROS, temperature and washing ratio. The methods also have 3 oils: load, raffinated and dewaxed. All oils have density, IR and VI as characteristics. The load has the type of cut as an additional characteristic, while the raffinate and dewaxed have the respective yields of each process as a characteristic.

In a preferred embodiment of the present invention, the data are received in 2 spreadsheets, one containing runs of the dearomatization process with only one sample of load oil for each cut, called the dearomatization spreadsheet, and one containing several runs of the entire process of the route, called the dewaxing spreadsheet. Therefore, there is much more dearomatization data than dewaxing data. Table 4 below shows the distribution of cut data in the database. Therefore, the cuts are well separated by IVC, IRC and dC, so most models do not need to be informed of the cut explicitly.

The IR, density and VI measurements are standardized in ASTM, respectively D1218, D4052 and D2270 (ASTM-2016). The measurement error of each variable for measurements made by different people is given in ASTM standards according to Table 5 below. The yield does not have an associated error because it is not a standardized measurement, but the yield error is estimated to be ±1.

In general, for modeling purposes, it is assumed that manipulated variables have negligible error, but for simulation purposes, an acceptable range is sought in which the model error does not significantly interfere with the result. These ranges are described in Table 6.

TABLE 4

| Acronym | Cut | Amount of data |
|---|---|---|
| NP | Heavy Neutral | 70 |
| NM | Medium neutral | 65 |
| NL | Light neutral | 64 |
| BS | Bright stock | 45 |
| SPM | Spindle motor | 9 |
| SPB | White Spindle | 9 |
| DAO | Deasphalted | 4 |

TABLE 5
ASTM Standards Error

| | |
|---|---|
| Density | 0.00052 |
| IR | 0.00050 |
| VI | 2 |

TABLE 6

| Manipulated variable | Acceptable error range |
|---|---|
| T | 1 |
| RSO | 0.5 |

The correlation analysis is based on calculating the correlation between each variable and was performed for each cut. The analysis was divided by cuts to avoid the Yule-Simpson effect in which trends that appear in parts of the data are lost when the whole is analyzed (Simpson, 1951).

In the tests performed, analyses were also performed both to evaluate how the load properties are influenced by the cut and to predict the VI of the mixture from the data of the individual components.

The first analysis is to better elucidate the behavior of the models and explain why some do not need to receive the cut explicitly as an input variable. The hypothesis is that the cut is implicit in the load properties, density and VI. A small RF classification model was tested that tries to infer the type of sample cut from the input data, thus showing that the model can infer the type of cut from the load property data. Additionally, the stratification of certain operating conditions in terms of the type of cut was analyzed.

The second analysis aims to try to predict the characteristics of the mixtures from the individual components, in order to be able to use the models without needing a prior analysis of the load. The IR and density can be inferred with simple arithmetic means. Predicting the viscosity and/or VI is more complicated, as mentioned previously. Approaches based on means were tested, such as Kendall-Monroe and Arrehnius, using both the VI and the viscosities themselves, and parameter estimation with an optimization method, using the function a*IV₁^{b}+c*IV₂^{d} and optimizing a, b, c, d.

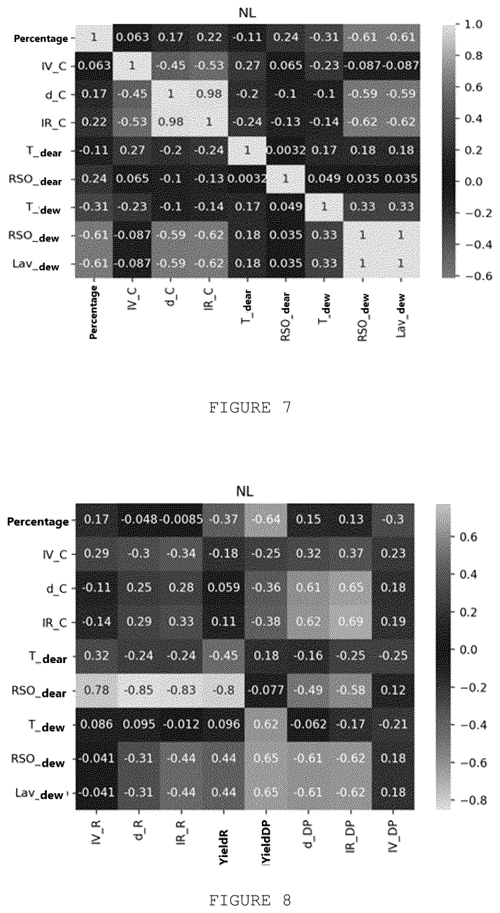

The clearest factor in the data analysis is the strong correlation between the IR and density, which was expected from the cutting literature. In general, the models worked very well for predicting IR and density, as seen in FIGS. 7 and 8, which show correlation matrices for the NL cut.

In general, the variable most related to the properties of the dearomatization products is RSOdear, as can be seen in FIG. 7. Mainly because it is the manipulated variable with the greatest variance in the data. Most of the other variables remain constant in most runs. Mainly the wash ratio in dewaxing, which alternates between values 1.5 and 2, including in some cuts it alternates exactly like RSOdew-percentage of mixture with a naphthenic oil, in general it also varies between 0 and 20%, reaching 30% on few occasions. IVC is also a variable of interest, as it has no correlation with the properties of the load and may contain information about the load that IRC and dC do not have.

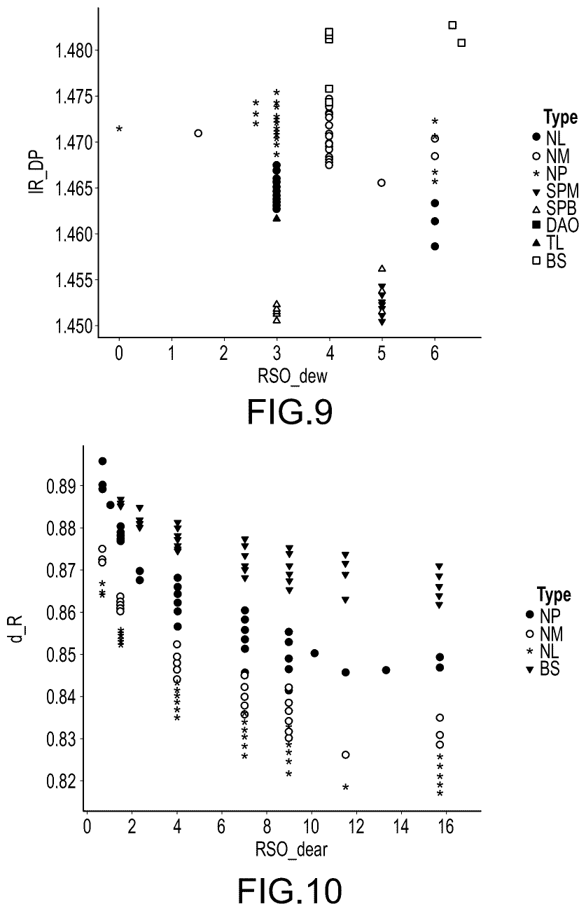

The analysis of the data from the dearomatization spreadsheet allowed us to visualize the effect of RSOdear and Tdear over a wide range of values. Some variables have a nonlinear effect, as can be seen in FIG. 9. This motivated the creation of expansion terms as data input. For example, instead of using RSOdear directly, log(RSOdear) and RSOdear² could also be used.

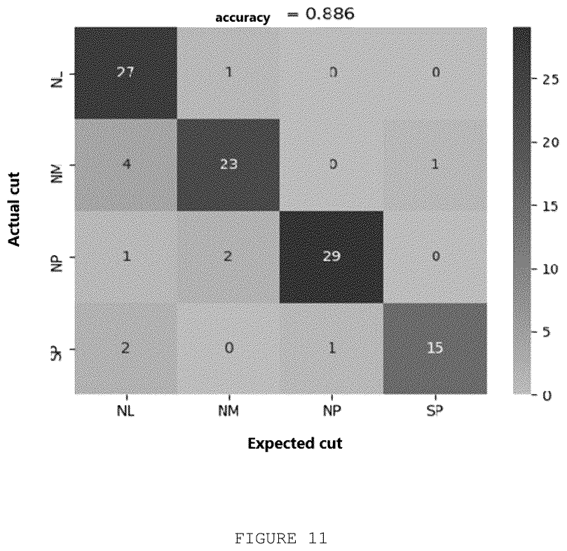

Some operating conditions are also very concentrated around certain types of cut. In FIG. 10, it can be seen that for NL and NP cuts, the dewaxing RSO is almost always 3, and for NM, the dewaxing RSO is almost always 4. This indicates that the inverse models should predict this variable very well. On the other hand, if the operator wants to test the product of these cuts under other operating conditions, the model will probably produce an unreliable result. As can be seen in FIG. 11, the model can predict the cut from density and IVC with 88.6% accuracy, presenting greater difficulty with the light neutral.

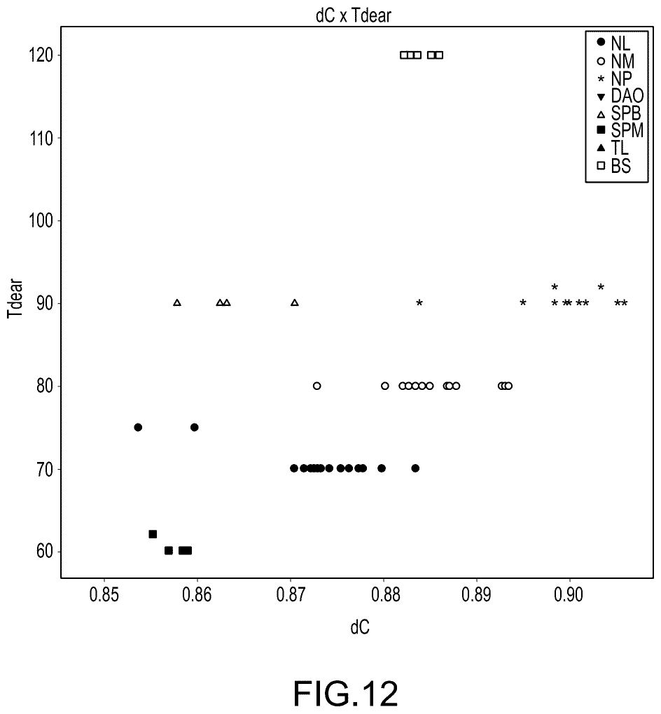

As can be seen in FIG. 12, there is a strong stratification of the data by cut in Tdear, these results demonstrate that the type of cut is implicit within the load property data and operating conditions and often does not need to be explicitly informed.

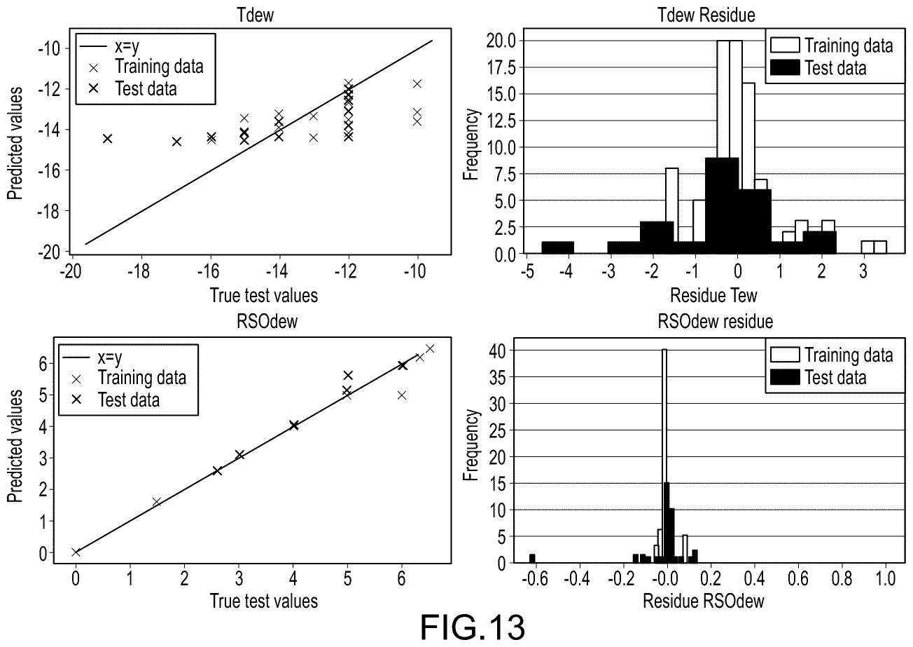

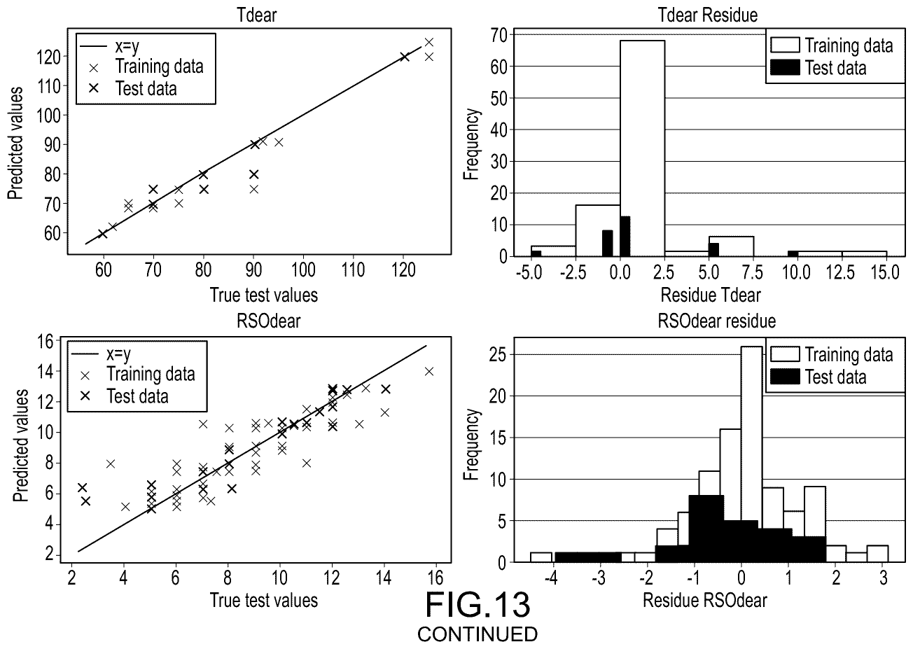

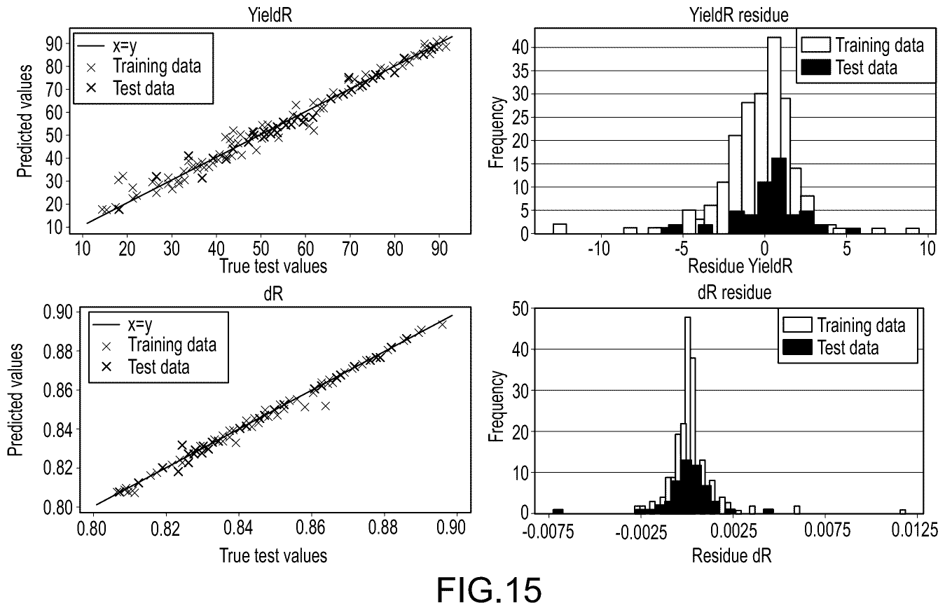

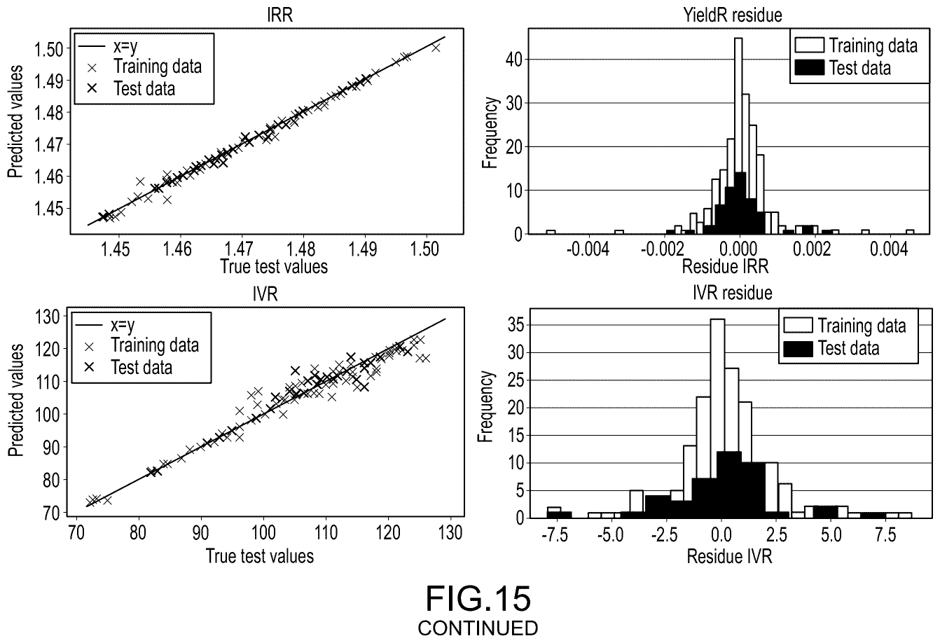

FIG. 13 and Tables 7 and 8 below show the results obtained by the best model selected to calculate the variables of interest. Table 7 shows the machine learning technique, and the inputs used in the best individual model, along with the R² values and the root mean square error (RMSE) for the training and test data sets. Table 7 shows the percentage of samples correctly predicted, considering an acceptable error range for the temperature of ±1° C. and for the RSO of ±0.5. FIG. 15 shows the prediction and residual plots for each variable.

TABLE 7
Best Individual Model

| | R² | R² | RMSE | RMSE | | |
|---|---|---|---|---|---|---|
| | Training | Test | Training | Test | Model | Inputs |
| Tdew | 0.608 | 0.456 | 0.952 | 1.444 | GB | d_{C}, IV_{DP}, Cut |
| RSOdew | 0.990 | 0.981 | 0.109 | 0.141 | GB | d_{C}, IV_{DP}, % |
| Tdear | 0.977 | 0.965 | 2.441 | 2.909 | RF | d_{C}, log(IV_{DP}), IV_{C} |
| RSOdear | 0.778 | 0.825 | 1.151 | 1.318 | GB | d_{C}, Cut |

TABLE 8
Values with acceptable error %
T = ±1
RSO = ±0.5

| | Training | Test |
|---|---|---|
| Tdew | 79 | 67 |
| RSOdew | 99 | 96 |
| Tdear | 82 | 79 |
| RSOdear | 49 | 42 |

It can be seen that the results for RSOdew and Tdear are excellent, with quite satisfactory R² values. In the prediction graphs, the samples are overlapped, so that the 120 samples do not appear to be the same but are depicted. In the case of RSOdew, practically all samples were predicted correctly, and the residuals are very low. In Tdear, more than 80% of the samples were predicted within the desired range.

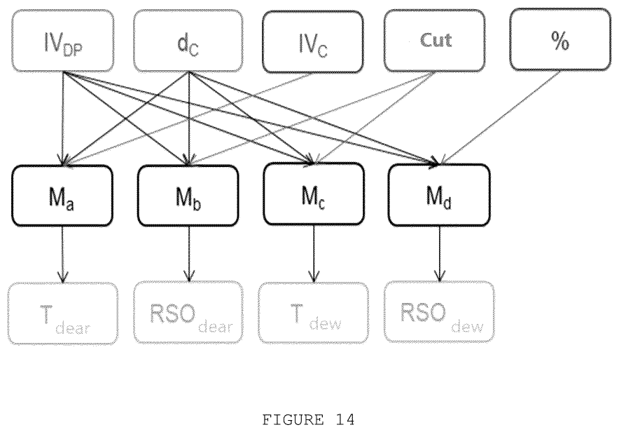

The Tdew and RSOdear models can still be improved, but they perform a prediction within the expected range. Although the R² values are higher in RSOdear, a smaller number of samples were predicted within the expected error range. More than 75% of the samples had Tdew predicted correctly. Considering the current best individual models, the optimization method structure is illustrated as shown in FIG. 14.

Similarly to FIG. 13 and Tables 7 and 8, FIG. 15 and Tables 9 and 10 show the results of the best models for predicting the dearomatization variables. In this case, the ranges considered acceptable for error are: dR=±0.00052, IRR=±0.00050, IVR=±2 and YieldR=±1.

TABLE 9
Best Individual Model

| | R² | R² | RMSE | RMSE | | |
|---|---|---|---|---|---|---|
| | Training | Test | Training | Test | Model | Inputs |
| YieldR | 0.982 | 0.981 | 2.450 | 2.245 | GB | d_{C}, Tdear, RSOdear, |
| | | | | | | 1/RSOdear |
| d_{R} | 0.995 | 0.995 | 0.001 | 0.001 | GB | d_{C}, Tdear, |
| | | | | | | RSOdear |
| IR_{R} | 0.995 | 0.996 | 0.001 | 0.001 | GB | d_{C}, Tdear, 1/RSOdear |
| IV_{R} | 0.962 | 0.930 | 2.236 | 2.481 | GB | d_{C}, Tdear, |
| | | | | | | Tdear*log(RSOdear) |

TABLE 10
Values with acceptable error %
d = ±0.00052 IR = ±0.00050
IVR = ±2 Yield = ±1

| | Training | Test |
|---|---|---|
| YieldR | 48 | 53 |
| d_{R} | 55 | 49 |
| IR_{R} | 66 | 77 |
| IV_{R} | 77 | 74 |

In the tests performed, all models for this stage had extremely satisfactory results, with R² values above 0.93 for all data sets and variables. In the case of dR and IRR, these values reached more than 0.99. However, observing Table 12 below, it can be seen that these two variables did not have such a significant number of predicted samples within the acceptable range for error.

This may be due to the reference values used to define the acceptable range. They were based on ASTM standards for acceptable reproducibility with 95% confidence. These values are very strict, and the data provided is already subject to this experimental measurement variation, so the prediction error would be a propagation of this.

The graphs presented demonstrate an excellent predictive capacity, and well-distributed residues following a normal pattern. Both confirm that the dearomatization variables are being predicted adequately.

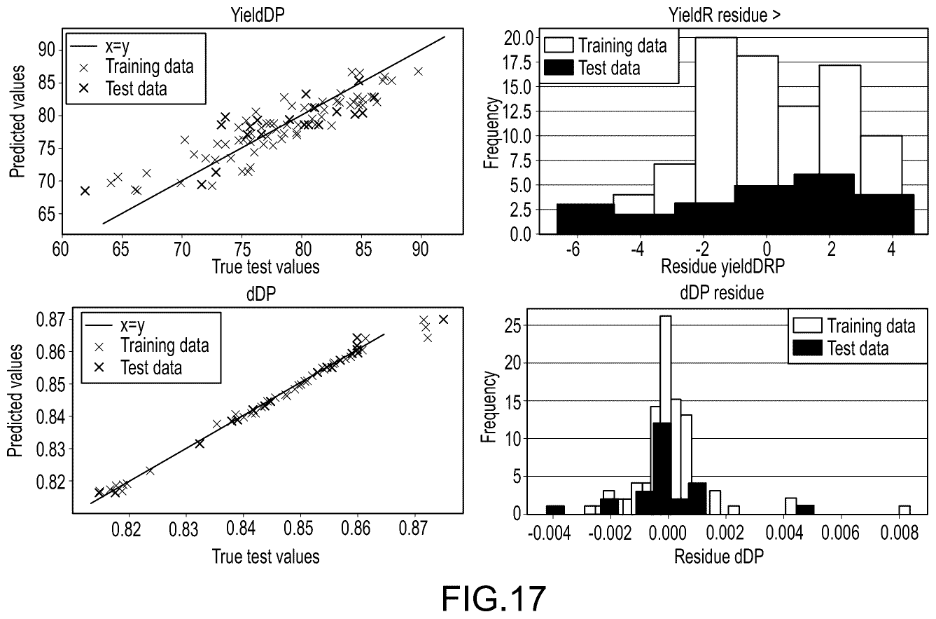

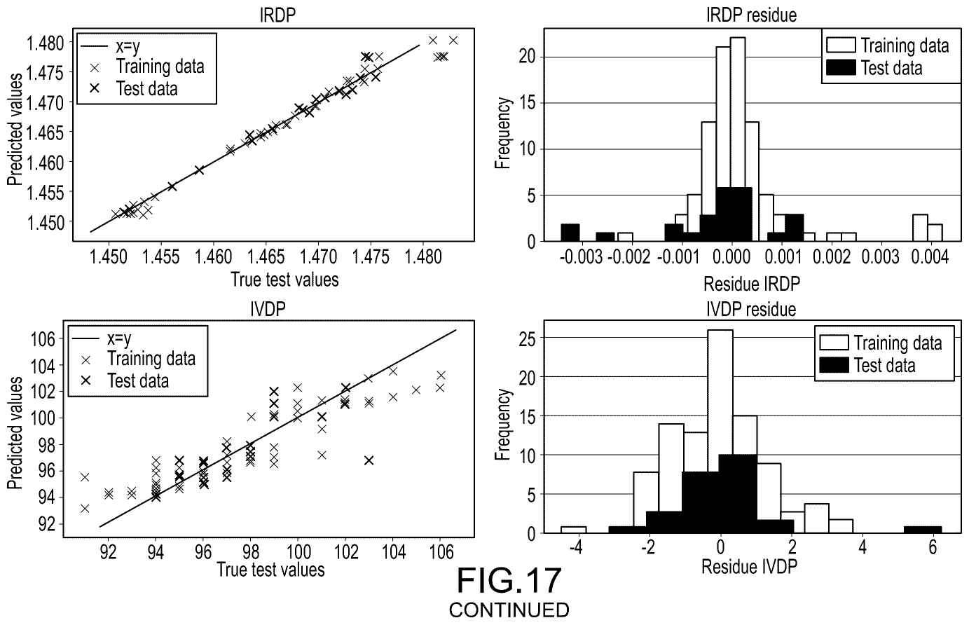

Once again, FIG. 17 and Tables 11 and 12 below are analogous to those presented in the previous sections, illustrating the results obtained in the tests for the best model in predicting each dewaxing variable. The acceptable error ranges are the same as for the dearomatization variables.

As in dearomatization, dDP and IRDP had excellent results, with excellent prediction graphs and well-distributed residuals, in addition to very high R² values, above 0.96. However, only between 50% and 70% of the samples were considered as predicted within the expected range.

In the case of IVDP, the results are satisfactory, with good noise distribution and a good margin of accuracy. For YieldDP, the data are more dispersed, and the model is not as suitable. Only 25% of the samples are usually predicted within the acceptable range. For both variables, it was observed throughout the modeling that the addition of the mixture percentage as an input variable improved the predictive capacity of the developed models.

TABLE 11
Best Individual Model

| | R² | R² | RMSE | RMSE | | |
|---|---|---|---|---|---|---|
| | Training | Test | Training | Test | Model | Inputs |
| YieldDP | 0.791 | 0.657 | 2.371 | 3.138 | GB | d_{R}, Tdew, RSOdew, |
| | | | | | | Tdew*RSOdew, % |
| d_{DP} | 0.991 | 0.988 | 0.001 | 0.001 | GB | d_{R}, Tdew, |
| | | | | | | log(RSOdew) |
| IR_{DP} | 0.978 | 0.967 | 0.001 | 0.001 | GB | d_{R}, Tdew, RSOdew, |
| | | | | | | Tdew*RSOdew |
| IV_{DP} | 0.836 | 0.566 | 1.378 | 1.655 | GB | d_{R}, Tdew, RSOdew, |
| | | | | | | log(RSOdew) , % |

TABLE 12
Values with acceptable error %
d = ±0.00052 IR = ±0.00050
IVR = ±2 Yield = ±1

| | Training | Test |
|---|---|---|
| YieldDP | 26 | 22 |
| d_{DP} | 60 | 52 |
| IR_{DP} | 69 | 60 |
| IV_{DP} | 84 | 88 |

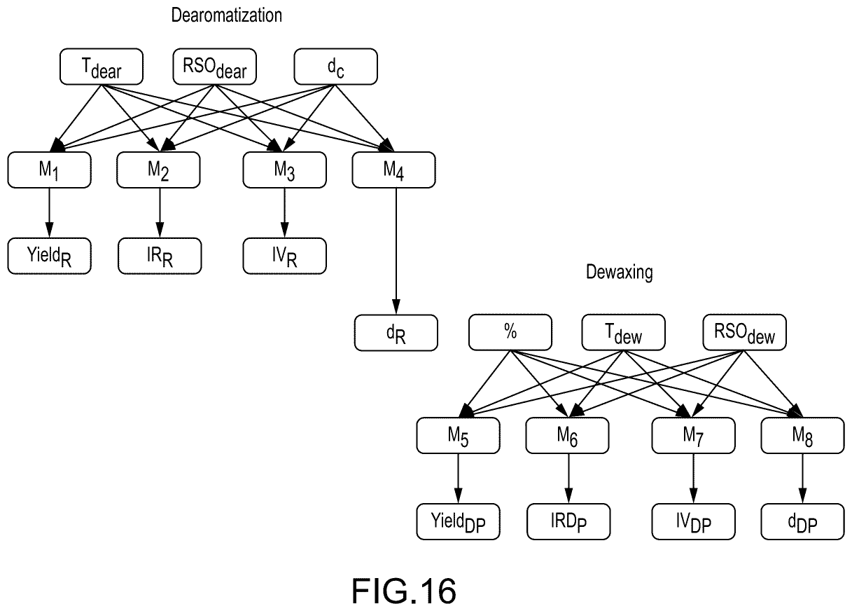

Considering the current best individual models, the prediction framework for dearomatization and dewaxing is illustrated in FIG. 16. Where the hyperparameters related to each final model are summarized in Table 13 below. All models are of the GradientBoostinRegressor type, except Tdear.

TABLE 13

| Model | Hyperparameters |
|---|---|
| YieldR | max_depth = 5, min_leaf = 4, n_estimators = 98 |
| d_{R} | max_depth =, 7min_leaf = 3, n_estimators = 73 |
| IR_{R} | max_depth = 7, min_leaf = 3, n_estimators = 68 |
| IV_{R} | max_depth = 7, min_leaf = 2, n_estimators = 49 |
| YieldDP | max_depth = 5, min_leaf = 7, n_estimators = 58 |
| d_{dp} | max_depth = 10, min_leaf = 5, n_estimators = 100 |
| IR_{dp} | max_depth = 5, min_leaf = 8, n_estimators = 100 |
| IV_{dp} | max_depth = 5, min_leaf = 7, n_estimators = 86 |
| Tdew | max_depth = 5, min_leaf = 10, n_estimators = 33 |
| RSOdew | max_depth = 7, min_leaf = 1, n_estimators = 40 |
| Tdear | max_depth = 5, min_leaf = 1, n_estimators = 1* |
| RSOdear | max_depth = 10, min_leaf = 3, n_estimators = 39 |

The model for Tdear is actually a decision tree and not a forest, since n_estimators=1. However, for reasons of code compatibility, the model is an object of the RandomForestRegressor class.

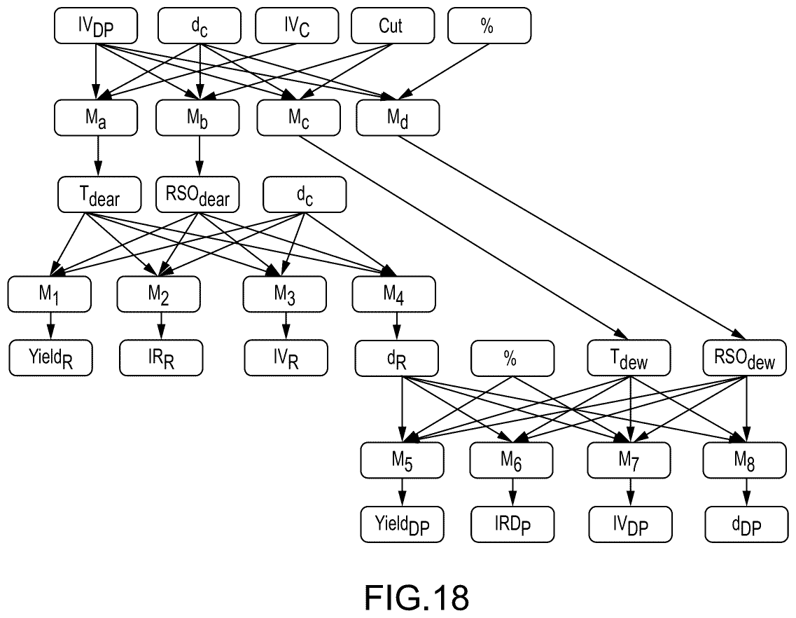

As previously mentioned, the optimization method tab receives the necessary input variables from the user and calculates the recommended operating conditions. In addition, this tab also uses these calculated conditions as intermediate variables, applying them as input to the dearomatization and dewaxing prediction models. In this way, the step yield and product properties are also provided as results. This strategy is illustrated in FIG. 18 and allows the best decision regarding the mixture between paraffinic and naphthenic oils so that the products can be specified. The calculation of all variables of interest can be done using only five variables provided by the user: dC, IVC, mixture percentage (%), type of cut (Cut) and the desired IVDP.

This structure leads us to believe that there will naturally be an error propagation, since the intermediate variables will already have their own margin of error. Theoretically, the predicted output variables of dearomatization and dewaxing will have their margins widened.

To simulate the behavior of the graphical interface optimization method guide, 97 samples were chosen according to the availability of all the necessary input, intermediate and output variables. The structure of FIG. 18 was applied, and Table 14 below shows the results obtained in this evaluation, in which the same acceptable error intervals presented previously for these variables were used. In Table 14, there are R² and RMSE values for the prediction of dearomatization and dewaxing in the graphical interface optimization method guide.

TABLE 14
Best Individual Model

| | | | % | | |
|---|---|---|---|---|---|
| | | | Acceptable | | |
| | R² | RMSE | Error | Model | Inputs |
| YieldR | 0.877 | 3.797 | 35 | GB | d_{C}, Tdear, |
| | | | | | RSOdear, |
| | | | | | 1/RSOdear |
| d_{R} | 0.971 | 0.002 | 33 | GB | d_{C}, Tdear, |
| | | | | | RSOdear |
| IR_{R} | 0.967 | 0.001 | 35 | GB | d_{C}, Tdear, |
| | | | | | 1/RSOdear |
| IV_{R} | 0.399 | 3.865 | 56 | GB | d_{C}, Tdear, |
| | | | | | Tdear*log |
| | | | | | (RSOdear) |
| YieldDP | 0.131 | 4.607 | 14 | GB | d_{R}, Tdew, |
| | | | | | RSOdew, |
| | | | | | Tdew*RSOdew, % |
| d_{DP} | 0.954 | 0.003 | 26 | GB | d_{R}, Tdew, |
| | | | | | log (RSOdew) |
| IR_{DP} | 0.947 | 0.002 | 32 | GB | d_{R}, Tdew, |
| | | | | | RSOdew, |
| | | | | | Tdew*RSOdew |
| IV_{DP} | 0.710 | 1.731 | 75 | GB | d_{R}, Tdew, |
| | | | | | RSOdew, |
| | | | | | log (RSOdew), % |

In general, the R² values and the percentage of correctly predicted samples decrease, while the RMSE values increase. This was the expected result, given the error propagation mentioned.

The variables dR, IRR, dDP and IRDP continue to have excellent results, with R² values above 0.94 and low RMSE values. This is important, since dR is used as an intermediate variable, being applied as input in all dewaxing models. If it is well predicted, the error propagation tends to be smaller.

While YieldR and IVDP continue to have reasonable results, YieldDP and IVR worsened considerably. However, in general it was observed that the results are in the correct order of magnitude.

The simulation guide, as previously explained, receives the necessary input variables from the user and calculates the properties of the raffinate and dewaxed product, in addition to the dearomatization and dewaxing yield. The strategy used in this guide is the same as that shown in FIG. 14.

The calculation of all variables of interest in this guide can be done using only six variables provided by the user: the load density (dc), the percentage of the mixture (%) and the operating conditions used (Tdear, RSOdear, Tdew and RSOdew).

All models for this stage had extremely satisfactory results, with R² values above 0.93 for all data sets and variables. In the case of dR and IRR, these values reached more than 0.99. However, observing Table 10, it can be seen that these two variables did not have such a significant number of predicted samples within the acceptable range for error.

This may be due to the reference values used to define the acceptable range. They were based on ASTM standards for acceptable reproducibility with 95% confidence. These values are very strict and relative to the experimental stage. The data provided are already subject to this experimental measurement variation, so the prediction error would be a propagation of this. The graphs presented demonstrate an excellent predictive capacity, and residues well distributed following a normal pattern. Both confirm that the dearomatization variables are being predicted adequately.

As previously mentioned, the date and time of the analysis are printed in the text, to facilitate the search, as well as the fact that it was a calculation performed in the simulation or optimization method tab. Furthermore, in the event of a problem, the added message is displayed to alert the user.

1- A method for predicting mixtures between petroleum products for processing in solvent route, obtaining group I lubricant base oils, comprising:
(a) creating a representative database of a production process of group I lubricant base oils, specifically of dearomatization and dewaxing steps;
(b) statistically analyzing said database and variables of said process, determining a combination of input variables for a prediction of output variables, said variables being a yield of each said dearomatization and dewaxing step, and properties of a raffinate and a dewaxed product;
(c) developing models from machine learning tools inferring said properties of raffinate and dewaxed product, such as density, refractive index and viscosity, as well as yield of each step, from properties of a load and operational variables; and
(d) developing a retraining module, which is activated as new data is acquired, allowing the insertion of new data, retraining of models, and the use of the retrained models as well as the original ones, which are no longer discarded if there is retraining.

2- The method of claim 1, wherein in step (d), activating said retraining module comprises:
extracting statistics from old data, such as minimum, maximum, mean and standard deviation;
analyzing new data, if the data is of the same type, evaluating whether all variables exist;
checking statistics made, whether the values are within a minimum and a maximum of said old data;
checking a mean and a covariance based on a Mahalabonis distance; and
obtaining clean data without missing variables or different descriptive statistics, thus testing models with new data.

3- The method of claim 1, wherein step (d) comprises a retraining module composed of an alternative database, a valid database and an old database,
wherein said alternative database is composed of data whose analyzed descriptive statistics are different from the descriptive statistics of data previously used;
wherein said valid database is composed of data whose analyzed descriptive statistics are similar to the descriptive statistics of data previously used; and
wherein said old database is composed of initial data used in the development of the tool.

4- The method of claim 3, characterized in that, in said alternative database of the retraining module of step (d), if the amount of data is equal to or greater than a number N, the amount of alternative data is evaluated.

5- The method of claim 1, further comprising predicting a limit of 30% in a pilot plant for a mixture between a type of oil A and a type of oil B.

6- A method for optimizing mixtures between petroleum products for processing in the solvent route, obtaining group I lubricant base oils, comprising:
(a) creating a representative database of a production process of group I lubricant base oils, specifically of dearomatization and dewaxing steps;
(b) statistically analyzing said database and variables of said process, determining a combination of input variables for an optimization of output variables of interest, that is, operating conditions of said process in each step;
(c) developing models from machine learning tools defining which values of the manipulated variables generate a raffinate and a dewaxed product with the desired properties, defined according to the characteristics of a product that is intended to be obtained, depending on its application; and
(d) developing a retraining module, which is activated as new data is acquired, allowing the insertion of new data, retraining of models, and the use of the retrained models as well as the original ones, which are no longer discarded if there is retraining.

7- The method of claim 6, wherein if a mixture of petroleum products results in valid operational conditions and yields, that is, physically and operationally viable values, it is considered that this mixture can be tested in the pilot plant with conditions similar to those optimized by the method.

8- A non-transitory computer-readable storage medium comprising instructions stored therein, characterized in that the instructions, when read by a computer, cause the computer to execute the steps of the method as defined in claim 1.

9- A non-transitory computer-readable storage medium comprising instructions stored therein, characterized in that the instructions, when read by a computer, cause the computer to execute the steps of the method as defined in claim 6.
